(19) United States
(12) Patent Application Publication
(10) Pub. No.: US 2017/0196441 A1
(43) Pub. Date: Jul. 13, 2017

(54) Liquid Ejecting Unit, Liquid Ejecting Device, Endoscopic Device, and Medical Device

(71) Applicant: Seiko Epson Corporation, Tokyo (JP)

(72) Inventors: Hideki Kojima, Matsumoto-shi (JP); Hirokazu Sekino, Chino-shi (JP)

(21) Appl. No.: 15/325,217

(22) PCT Filed: Jun. 23, 2015
(86) PCT No.: PCT/JP2015/003140

§ 371 Date: Jan. 10, 2017

(30) Foreign Application Priority Data
Jul. 10, 2014 (JP) 2014-142038

(51) Int. Cl. A61B 1/015

(52) U.S. Cl. CPC A61B 1/015

(57) ABSTRACT

In a case of being used in combination with an endoscope, a liquid chamber needs to be inserted into the endoscope. A liquid ejecting unit is used in combination with the endoscope including an elongated tubular portion for being inserted into a body and a grip portion connected to the tubular portion. In a case where the liquid ejecting unit is combined with the endoscope, the liquid chamber is disposed outside the endoscope.

### TECHNICAL FIELD

The present invention relates to liquid ejection.

### BACKGROUND ART

In a liquid ejecting unit for ejecting a pulsating flow, a configuration is known in which a liquid chamber for generating the pulsating flow is disposed in the vicinity of an opening portion of an ejecting tube (for example, refer to PTLS 1, 2, and 3).

### CITATION LIST

### Patent Literature

PTL 1: JP-A-2012-223266

PTL 2: JP-A-2012-187291

PTL 3: JP-A-2009-136519

### SUMMARY OF INVENTION

### Technical Problem

In view of the related art, the invention aims to solve a problem that a liquid chamber needs to be inserted into an endoscope in a case where the liquid chamber is used in combination with the endoscope. If the liquid chamber is disposed in the vicinity of the opening portion of the ejecting tube as disclosed in the related art, a problem arises in that usability of the liquid ejecting unit becomes poor when the liquid ejecting unit is used in combination with the endoscope, or in that design constraints are imposed on the liquid chamber.

### Solution to Problem

The invention is made in order to solve the above-described problem, and can be realized by the following aspects.

(1) According to an aspect of the invention, there is provided a liquid ejecting unit which is used in combination with an endoscope including an elongated tubular portion for being inserted into a body and a grip portion connected to the tubular portion. The liquid ejecting unit includes a liquid chamber that is disposed outside the endoscope in a case where the liquid ejecting unit is combined with the endoscope; a pulsating flow generation unit that generates a pulsating flow in the liquid chamber; an opening portion for ejecting a liquid; and a connection channel that is flexible, and that connects the liquid chamber and the opening portion to each other. According to this aspect, in a case of being used in combination with an endoscope, the liquid chamber is not inserted into the endoscope. As a result, satisfactory usability of the liquid ejecting unit is achieved, and the liquid chamber is more freely designed.

(2) According to another aspect of the invention, there is provided a liquid ejecting unit which is used in combination with an endoscope including an elongated tubular portion for being inserted into a body and a grip portion connected to the tubular portion. The liquid ejecting unit includes a pulsating flow generation unit that generates a pulsating flow in a liquid chamber; an opening portion for ejecting a liquid; and a connection channel that is flexible, and that connects the liquid chamber and the opening portion to each other. A length of the connection channel is longer than a length of the tubular portion. According to this aspect, in a case of being used in combination with the endoscope, satisfactory usability is achieved. In a case of this aspect, the connection channel is longer than the tubular portion. Accordingly, even if the connection channel is inserted into the tubular portion, the connection channel protrudes from the tubular portion. Accordingly, the protruding portion can be gripped or operated. The opening portion is enabled to protrude from the tubular portion. Therefore, the above-described advantageous effect can be obtained.

(3) According to still another aspect of the invention, there is provided a liquid ejecting unit including a pulsating flow generation unit that generates a pulsating flow in a liquid chamber; an opening portion for ejecting a liquid; a connection channel that is flexible, and that connects the liquid chamber and the opening portion to each other; and a liquid supply channel that functions as a channel for supplying a liquid to the liquid chamber, and that communicates with the liquid chamber. At least one of a Young's modulus, an outer diameter, a wall thickness, and a Poisson's ratio is different between the connection channel and the liquid supply channel. According to this aspect, the connection channel and the liquid supply channel can be provided with suitable characteristics. The pulsating flow is not applied to the liquid flowing in the liquid supply channel by the pulsating flow generation unit. In contrast, the pulsating flow is applied to the liquid flowing in the connection channel by the pulsating flow generation unit. That is, in a case of this aspect, at least one of the Young's modulus, the outer diameter, the wall thickness, and the Poisson's ratio is different between the mutually different channels in which the pulsating flow generated by the pulsating flow generation unit is present or absent. As a result, the channels can have characteristics corresponding to the presence or absence of the pulsating flow generated by the pulsating flow generation unit. Therefore, the above-described advantageous effect can be obtained.

(4) In the above-described aspect, an increment of a cross-sectional area of a channel due to predetermined internal pressure loaded to the connection channel may be smaller than an increment of a cross-sectional area of a channel due to the predetermined internal pressure loaded to the liquid supply channel. According to this aspect, the connection channel and the liquid supply channel can be provided with more suitable characteristics. In order for the connection channel to propagate the pulsating flow generated by the pulsating flow generation unit to the ejecting tube without attenuating the pulsating flow, it is preferable that the connection channel has a small variation of the cross-sectional area which results from a variation in the internal pressure. On the other hand, there is no reason that the liquid supply channel needs to propagate the pulsating flow. Accordingly, it is not necessary to reduce the increment of the cross-sectional area which results from the variation in the internal pressure. Instead, it is preferable to manufacture the channel by giving priority to reduced channel resistance or cost reduction. According to this aspect, these characteristics are easily achieved.

(5) In the above-described aspect, a volume of the liquid chamber can be changed, and the pulsating flow generation unit may include a volume change portion which can change the volume of the liquid chamber. According to this aspect, the changed volume enables the pulsating flow to be applied thereto.

(6) In the above-described aspect, the pulsating flow generation unit may include an air bubble generation portion for generating air bubbles inside the liquid chamber. According to this aspect, the generated air bubbles enable the pulsating flow to be applied thereto.

The invention can be realized by adopting various aspects. For example, the invention can be realized by adopting aspects such as a liquid ejecting device including a liquid supply mechanism and a control device, an endoscopic device including the liquid ejecting unit, and a medical device employing both of these.

### BRIEF DESCRIPTION OF DRAWINGS

FIG. 1 is a configuration diagram of an endoscopic device.

FIG. 2 is a configuration diagram of a liquid ejecting unit.

FIG. 3 is a sectional view of an internal configuration of a pulsating flow generation unit.

FIG. 4 is a view illustrating a waveform of a drive voltage applied to a piezoelectric element.

FIG. 5 is a view illustrating a corresponding relationship between a waveform of a drive voltage and a state of diaphragm deformation.

FIG. 6 is a perspective view of an endoscope.

FIG. 7 is an enlarged view of a distal end portion of the endoscope.

FIG. 8 is a configuration diagram of an endoscopic device (Embodiment 2).

FIG. 9 is a configuration diagram of a liquid ejecting unit (Embodiment 2).

FIG. 10 is a sectional view of an internal configuration of a pulsating flow generation unit (Embodiment 2).

### DESCRIPTION OF EMBODIMENTS

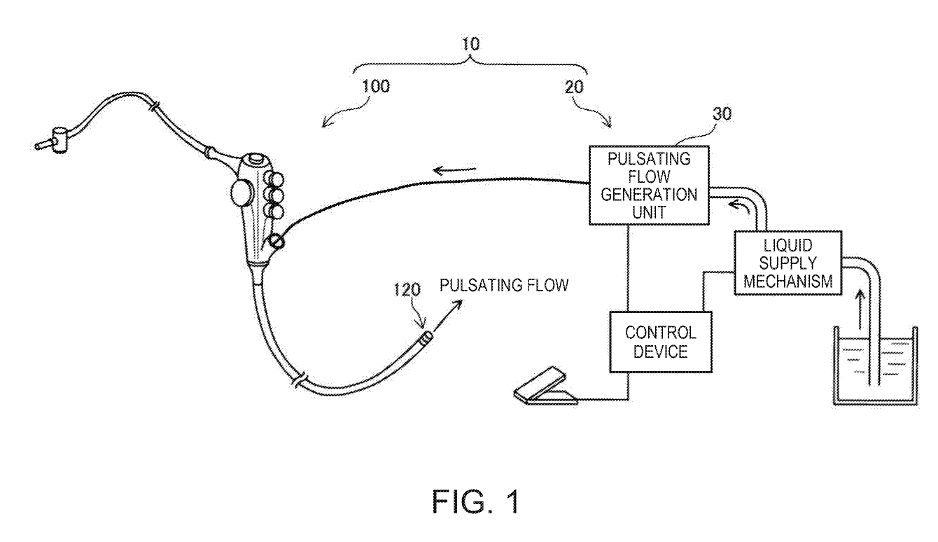

Embodiment 1 will be described. FIG. 1 is a configuration diagram of an endoscopic device 10. The endoscopic device 10 is configured so that a liquid ejecting device 20 is combined with an endoscope 100. The endoscopic device 10 is used as a medical device. The endoscopic device 10 has a function to provide an operator with an image inside a body of a patient, and a function to excise a lesion area by ejecting a liquid in a pulse shape to the lesion area from an ejecting tube 55 disposed inside a distal end portion 120. According to the present embodiment, the patient is a person.

As illustrated in FIG. 1, a pulsating flow generation unit 30 is disposed outside the endoscope 100 in a state where the liquid ejecting device 20 is combined with the endoscope 100.

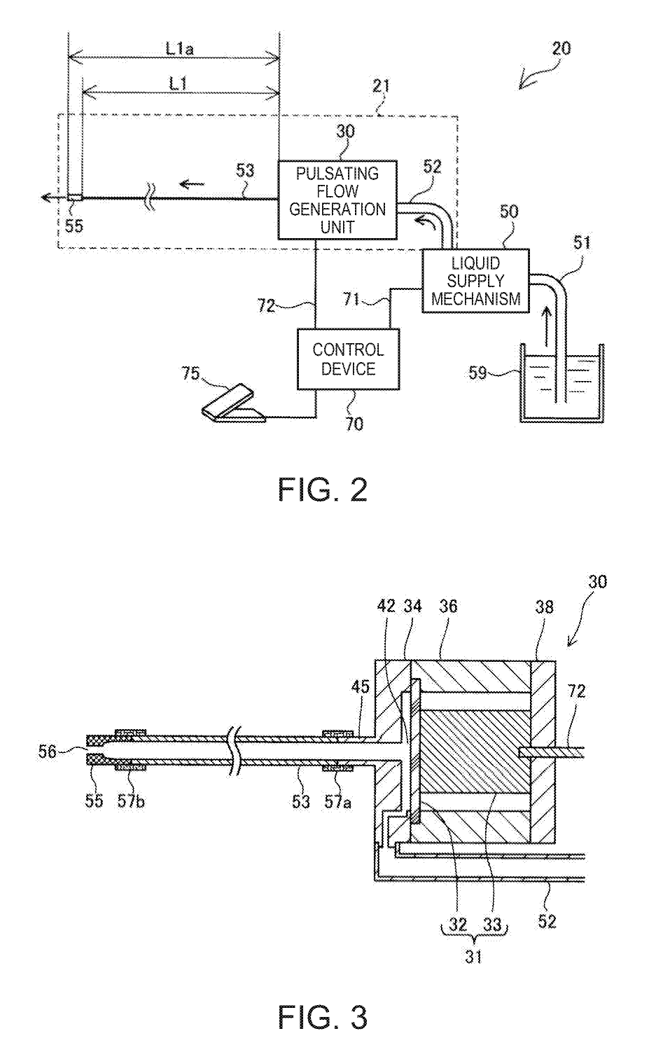

FIG. 2 illustrates the liquid ejecting device 20. The liquid ejecting device 20 includes the pulsating flow generation unit 30, a liquid supply mechanism 50, a channel 51, a liquid supply channel 52, a connection channel 53, a liquid container 59, a control device 70, a signal line 71, a signal line 72, and a foot switch 75. The pulsating flow generation unit 30, the liquid supply channel 52, the connection channel 53, and the ejecting tube 55 configure the liquid ejecting unit 21. The liquid ejecting unit 21 is a module replaced for every surgery. For the purpose of this replacement, the liquid supply channel 52 is detachably connected to the liquid supply mechanism 50, and the signal line 72 is detachably connected to the pulsating flow generation unit 30.

The liquid supply mechanism 50 aspirates a liquid stored in the liquid container 59 via the channel 51, and supplies the liquid to the pulsating flow generation unit 30 via the liquid supply channel 52. The liquid is a physiological saline solution. The liquid supply channel 52 is formed of polypropylene, and is flexible. The pulsating flow generation unit 30 generates a pulsating flow in the supplied liquid. The liquid having the pulsating flow generated therein is ejected from the ejecting tube 55 via the connection channel 53.

The control device 70 controls an operation of the pulsating flow generation unit 30 and the liquid supply mechanism 50. When actuated, the control device 70 always transmits a signal for supplying the liquid to the liquid supply mechanism 50 via the signal line 71. While the foot switch 75 is stepped on, the control device 70 transmits a signal for generating the pulsating flow to the pulsating flow generation unit 30 via the signal line 72.

The connection channel 53 is formed of a PEEK (registered trademark) resin. Whereas the PEEK resin is an insulator and flexible, the PEEK resin has a high Young's modulus. The ejecting tube 55 is made of stainless steel, and is a nozzle disposed in a distal end of the connection channel 53.

The pulsating flow is generated in the liquid supplied to the ejecting tube 55. Accordingly, as described above, the liquid is ejected in a pulse shape. The ejecting in the pulse shape described herein means that the liquid is ejected in a state resulting from variations in a flow rate or a flow velocity, and is not limited to a fact that the liquid is repeatedly ejected and stopped. That is, the ejecting in the pulse shape includes various ejecting forms such as a form in which the ejecting is completely interrupted between the injection and the injection and a form in which a low pressure flow is present even during the injection.

In order to realize the ejecting in the above-described pulse shape, it is preferable to design the connection channel 53 so that the pulsating flow is not attenuated as possible. In order to minimize the attenuation of the pulsating flow, it is preferable that the connection channel 53 is less deformed due to the pulsating flow. Hereinafter, as a representative value relating to the deformation of the connection channel 53, a change of a cross-sectional area of the channel will be examined.

An increment ΔS of a cross-sectional area due to the pulsating flow is expressed by Equation (1) below.

ΔS=π{(rᵢₙ+u_{rMAX})²−(rᵢₙ+u_{rMIN})²} (1)

In Equation (1), rᵢₙ represents an internal radius in a case where the internal pressure is zero, u_{rMAX} represents an increment of the internal radius in a case of a maximum internal pressure P_{MAX}, and u_{rMIN} represents an increment of the internal radius in a case of a minimum internal pressure P_{MIN}. The increment of the internal radius represents an increment based on a case where the internal pressure is zero. If Equation (1) is organized, the organized one is expressed by Equation (2).

ΔS=π{2rᵢₙ(u_{rMAX}−u_{rMIN})−u_{rMAX}²−u_{rMIN}²} (2)

An increment uᵣ of an inner diameter is expressed by Equation (3) which is an equation relating to a thick cylinder, in a case where the internal pressure is Pᵢₙ.

$${u}_{r} = \frac{{P}_{in} {r}_{in}}{E} ( \frac{{r}_{out}^{2} + {r}_{in}^{2}}{{r}_{out}^{2} - {r}_{in}^{2}} + v ) \qquad ( 3 )$$

In Equation (3), E represents a Young's modulus, rₒᵤₜ represents an external radius in a case where the internal pressure is zero, and γ represents a Poisson's ratio. Based on Equation (2) and Equation (3), it can be described that the increment ΔS of the cross-sectional area is dependent on the Young's modulus E, the internal radius rᵢₙ, the external radius rₒᵤₜ, and the Poisson's ratio γ. The internal radius rᵢₙ is (external radius rₒᵤₜ-wall thickness). Accordingly, it can also be described that the increment ΔS of the cross-sectional area is dependent on the Young's modulus E, the external radius rₒᵤₜ, the wall thickness, and the Poisson's ratio γ.

Hereinafter, a calculation example of the increment AS of the cross-sectional area will be described using specific numerical values. The connection channel 53 is set to the Young's modulus E=3.6 GPa, the internal radius rᵢₙ=1 mm, the external radius rₒᵤₜ=2 mm, and the Poisson's ratio γ=0.4. The connection channel 53 is set to the maximum internal pressure P_{MAX}=1 MPa and the minimum internal pressure P_{MIN}=0 MPa. In this case, the increment ΔS of the cross-sectional area is calculated as 3.6×10⁻³ mm², based on Equation (2) and Equation (3). Accordingly, a volume increment of the channel per m in the flowing direction is 3.6 mm³.

Using the same calculation method, the increment ΔS of the cross-sectional area of the liquid supply channel 52 under the same pressure condition can be calculated. The liquid supply channel 52 is set to the Young's modulus E=1.5 GPa, the internal radius rᵢₙ=2 mm, the external radius rₒᵤₜ=3 mm, and the Poisson's ratio γ=0.4. Accordingly, compared to the connection channel 53, the Young's modulus E, the internal radius rᵢₙ, and the external radius rₒᵤₜ are different. On the other hand, the wall thickness (1 mm) and the Poisson's ratio γ are the same as those in the connection channel 53. In this case, the increment ΔS of the cross-sectional area is calculated as 5.03×10⁻² mm². Accordingly, a volume increment of the channel per m in the flowing direction is 50.3 mm³.

In Equation (2), if a second-order item of uᵣ is ignored, Equation (4) below is obtained.

ΔS=2πrᵢₙ(u_{rMAX}−u_{rMIN}) (4)

Normally, uᵣ is a smaller value than rᵢₙ. Accordingly, even if Equation (4) is used, substantially the same value as that in a case of using Equation (2) is calculated. If Equation (3) is substituted for Equation (4), Equation (5) below is obtained. ΔP included in Equation (5) represents the maximum internal pressure P_{MAX}—the minimum internal pressure P_{MIN}.

$$Δ S = 2 π {r}_{in}^{2} \frac{Δ P}{E} ( \frac{{r}_{out}^{2} + {r}_{in}^{2}}{{r}_{out}^{2} - {r}_{in}^{2}} + v ) \qquad ( 5 )$$

Based on Equation (5), it can be described that decreasing the internal radius rᵢₙ and the Poisson's ratio γ and increasing the external radius rₒᵤₜ and the Young's modulus E contribute to decreasing the increment ΔS of the cross-sectional area. Compared to the liquid supply channel 52, the connection channel 53 has the smaller external radius rₒᵤₜ. Since the internal radius rᵢₙ is small and the Young's modulus E is great, the increment ΔS of the cross-sectional area is small under the same pressure condition.

If the internal radius rᵢₙ is too small, the channel resistance inevitably increases. If the external radius rₒᵤₜ is too large, the channel is less likely to be inserted into the endoscope 100. If the Young's modulus E is too great, the flexibility becomes poor. The connection channel 53 according to the present embodiment employs the above-described values as a result that the connection channel 53 is designed in view of a balance therebetween.

As illustrated in FIG. 2, the length of the connection channel 53 is a length L1. The total length of the connection channel 53 and the ejecting tube 55 is a length L1a. As illustrated in FIG. 2, the lengths represent a value designed so that the connection channel 53 protrudes from the endoscope 100 in a state where the connection channel 53 and the ejecting tube 55 are inserted into the endoscope 100, and where the ejecting tube 55 is disposed in the vicinity of the distal end portion 120.

FIG. 3 is a sectional view of the pulsating flow generation unit 30, the connection channel 53, and the ejecting tube 55 including the opening portion 56 for ejecting the liquid. The pulsating flow generation unit 30 includes the pulsating flow generation unit 31 and the liquid chamber 42. As a volume change portion, the pulsating flow generation unit 31 includes a diaphragm 32 and a piezoelectric element 33.

As described above, in a state where the liquid ejecting device 20 is combined with the endoscope 100, the pulsating flow generation unit 30 is disposed outside the endoscope 100. Accordingly, in the state where the liquid ejecting device 20 is combined with the endoscope 100, the liquid chamber 42 included in the pulsating flow generation unit 30 is also disposed outside the endoscope 100.

The liquid chamber 42 is a space between a first case 34 and the diaphragm 32, and forms a channel between the liquid supply channel 52 and the connection channel 53. The diaphragm 32 is a disk-shaped thin metal plate. An outer peripheral portion of the diaphragm 32 is fixed by being pinched between the first case 34 and a second case 36. The first case 34, the second case 36, and a third case 38 (to be described later) are made of stainless steel.

As illustrated in FIG. 3, an outlet channel 45 communicating with the liquid chamber 42 is disposed so as to protrude from the first case 34. The outlet channel 45 and the connection channel 53 are connected to each other by a connection ring 57a. The connection channel 53 and the ejecting tube 55 are connected to each other by a connection ring 57b. The connection rings 57a and 57b are made of stainless steel, and are ring-shaped members. The connection ring 57a is fixed to each of the outlet channel 45 and the connection channel 53 by using an adhesive. The connection ring 57b is fixed to each of the connection channel 53 and the ejecting tube 55 by using an adhesive.

As illustrated in FIG. 3, the outlet channel 45, the connection channel 53, and the ejecting tube 55 have an equal inner diameter. However, the inner diameter of the ejecting tube 55 indicates the inner diameter in the connection portion between the connection channel 53 and the ejecting tube 55. In this manner, a step is not present in the channel from the outlet channel 45 to the connection portion of the ejecting tube 55, thereby restraining the pulsating flow from being attenuated.

The piezoelectric element 33 is an actuator operated by a drive voltage applied from the control device 70. The piezoelectric element 33 changes a volume of the liquid chamber 42 formed between the diaphragm 32 and the first case 34, thereby changing liquid pressure inside the liquid chamber 42. The piezoelectric element 33 is a multilayer piezoelectric element, in which one end is fixed to the diaphragm 32 and the other end is fixed to the third case 38.

If the drive voltage applied to the piezoelectric element 33 increases, the piezoelectric element 33 expands. The diaphragm 32 is pressed by the piezoelectric element 33, and is bent to the liquid chamber 42 side. If the diaphragm 32 is bent to the liquid chamber 42 side, the volume of the liquid chamber 42 decreases. The liquid inside the liquid chamber 42 is pressed out to the connection channel 53 from the liquid chamber 42.

On the other hand, if the drive voltage applied to the piezoelectric element 33 decreases, the piezoelectric element 33 contracts. The volume of the liquid chamber 42 increases. The liquid flows into the liquid chamber 42 from the liquid supply channel 52.

The drive voltage applied to the piezoelectric element 33 from the control device 70 is repeatedly turned on (maximum voltage) and turned off (0 V) at a predetermined frequency (for example, 400 Hz). Accordingly, the volume of the liquid chamber 42 repeatedly expands and contracts, thereby generating the pulsating flow in the liquid.

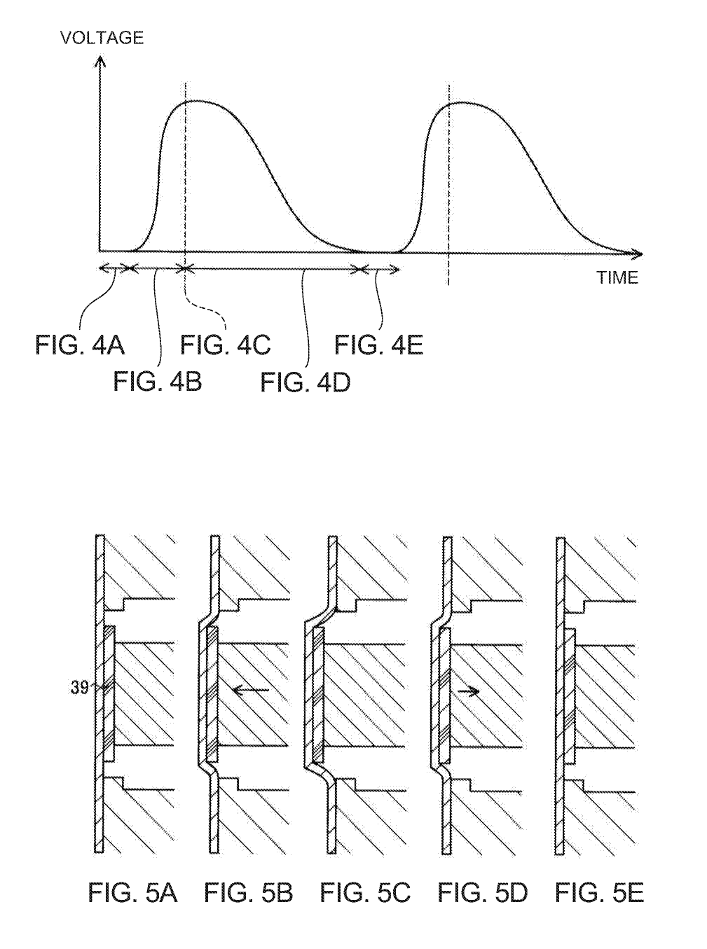

FIG. 4 illustrates an example of a waveform of the drive voltage applied to the piezoelectric element 33. In FIG. 4, the horizontal axis represents a time, and the vertical axis represents the drive voltage. One cycle of the waveform of the drive voltage is configured to include a rising period (b) during which the voltage increases, a time (c) during which the voltage is maximized, a falling period (d) during which the voltage decreases, and quiescent periods (a) and (e) during which the voltage is not applied.

The waveform during the rising period of the drive voltage shows a waveform corresponding to ½ cycle of a sin waveform which is offset in a positive voltage direction and whose phase is deviated as much as −90 degrees. The waveform during the falling period of the drive voltage shows a waveform corresponding to ½ cycle of the sin waveform which is offset in the positive voltage direction and whose phase is deviated as much as +90 degrees. Then, the cycle of the sin waveform during the falling period is larger than the cycle of the sin waveform during the rising period.

In a case where a magnitude of the drive voltage is changed, the maximum value of the waveform illustrated in FIG. 4 is changed. In addition, in a case where the frequency of the drive voltage is changed, the waveform during the rising period and the falling period is not changed, and the length of the quiescent period is changed.

FIG. 5 is a view illustrating a corresponding relationship between the waveform of the drive voltage and a state where the diaphragm 32 is deformed. In FIG. 5, a reinforcement member 39 is disposed between the piezoelectric element 33 and the diaphragm 32. During the quiescent period (a), the drive voltage is not applied. Accordingly, the piezoelectric element 33 does not expand, and the diaphragm 32 is not bent. During the rising period (b), the drive voltage increases. Accordingly, the piezoelectric element 33 expands, and the diaphragm 32 is bent to the liquid chamber 42 side, thereby decreasing the volume of the liquid chamber 42.

During the time (c), the drive voltage is maximized. Accordingly, the length of the piezoelectric element 33 is also maximized, and the volume of the liquid chamber 42 is minimized. During the falling period (d), the drive voltage decreases. Accordingly, the piezoelectric element 33 starts to recover the original size, and the volume of the liquid chamber 42 starts to recover the original size. During the quiescent period (e), the drive voltage is not applied. Accordingly, the piezoelectric element 33 recovers the original size, and the volume of the liquid chamber 42 recovers the original size. A series of operations illustrated from (a) to (e) is repeatedly performed, thereby generating the pulsating flow in the liquid inside the liquid chamber 42.

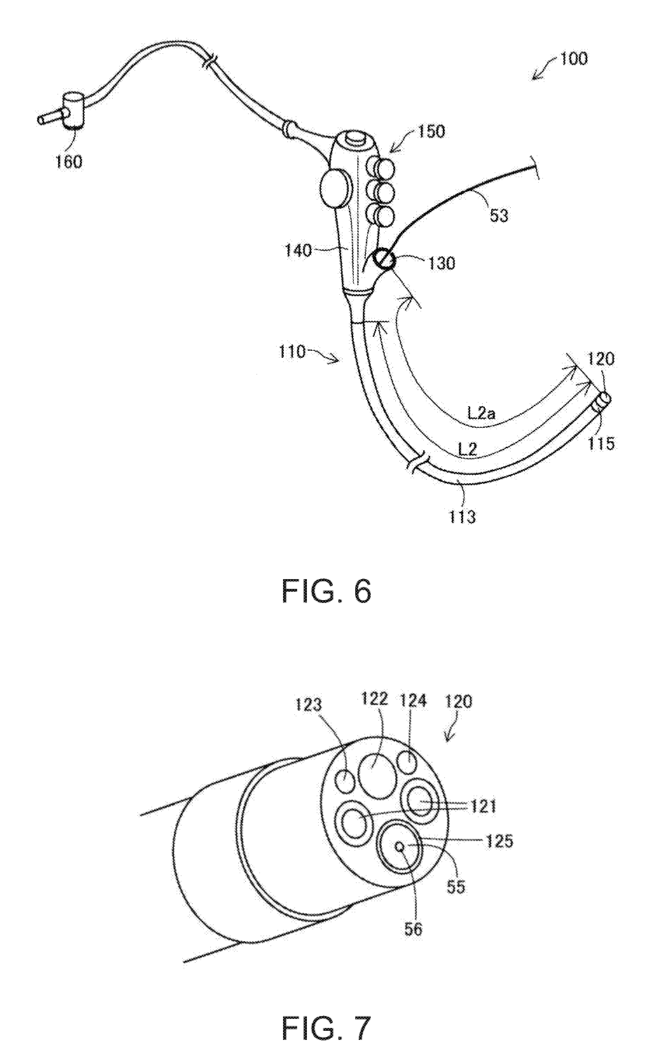

FIG. 6 is a perspective view of the endoscope 100. The endoscope 100 is a known flexible endoscope. FIG. 6 illustrates a state where the endoscope 100 is combined with the liquid ejecting device 20, specifically, a state where the connection channel 53 and the ejecting tube 55 are inserted into the endoscope 100.

The endoscope 100 includes a tubular portion 110, a connection channel insertion port 130, a grip portion 140, an operation unit 150, and a connection portion 160. The tubular portion 110 is a portion to be inserted into a body of a patient, and includes a flexible portion 113, a bend portion 115, and a distal end portion 120.

The flexible portion 113 illustrated in FIG. 6 is an elongated portion formed of a flexibly bending material, and the connection channel 53 and the ejecting tube 55 are inserted into the flexible portion 113. The bending direction of the bend portion 115 is changed by the operation of the operation unit 150. The connection channel insertion port 130 is an opening portion for inserting the connection channel 53 and the ejecting tube 55 into the tubular portion 110.

As illustrated in FIG. 6, the length of the tubular portion 110 is represented by a length L2, and the length from the connection channel insertion port 130 to the distal end portion 120 is represented by a length L2a. The length L2 is shorter than the length L2a. The length L2a is shorter than the length L1a, and is shorter than the length L1. Accordingly, the length L1 of the connection channel 53 is longer than the length L2 of the tubular portion 110. In addition, the length L2a is shorter than the length L1a. Accordingly, as described above, in a state where the connection channel 53 and the ejecting tube 55 are inserted into the endoscope 100, and where the ejecting tube 55 is disposed in the vicinity of the distal end portion 120, the connection channel 53 protrudes out from the connection channel insertion port 130. In this manner, the connection channel 53 is disposed so as to be easily gripped.

FIG. 7 is an enlarged view of the distal end portion 120. The distal end portion 120 has a light 121, an objective lens 122, an air and water supply port 123, an air and water intake port 124, and an opening portion 125. The objective lens 122 is disposed in order to observe the vicinity of the distal end portion 120, and is connected to an optical fiber (not illustrated) which passes through the inside of the flexible portion 113. The light 121 emits light for the observation.

The air and water supply port 123 is an opening portion for discharging air or the liquid such as physiological saline solution. The air and water intake port 124 is an opening portion for aspirating the surrounding air or the liquid. The opening portion 125 is disposed in order to expose the ejecting tube 55. FIG. 7 illustrates the inserted ejecting tube 55 and the opening portion 56 of the ejecting tube 55.

As illustrated in FIG. 6, the grip portion 140 is connected to the tubular portion 110, and is a portion to be gripped by an operator. The operation unit 150 is disposed on the upper portion of the grip portion 140. The operation unit 150 has a knob for operating the direction of the bend portion 115 and a button for operating air and water supply or air and water intake. The connection portion 160 is connected to illumination using the above-described light 121, a controller for realizing the air and water supply or the air and water intake, and a display for displaying an image captured by the objective lens 122.

The above-described liquid ejecting device 20 and endoscope 100 are combined with each other. In this manner, while an operator observes an image in the vicinity of a lesion area by inserting the tubular portion 110 into a body of a patient, the operator can excise the lesion area by ejecting the pulsating flow from the opening portion 56 of the ejecting tube 55 inserted into the tubular portion 110.

According to Embodiment 1 described above, at least the following advantageous effects can be obtained.

(A) In a state where the liquid ejecting device 20 is combined with the endoscope 100, the pulsating flow generation unit 30 including the liquid chamber 42 is disposed outside the endoscope 100. Accordingly, at least the following three advantageous effects are achieved.

(A-1) Even if an operator operates the connection channel 53 in order to adjust the position of the ejecting tube 55, the liquid chamber 42 does not move. Accordingly, the operator can easily operate the connection channel 53.

(A-2) The weight of the pulsating flow generation unit 30 including the liquid chamber 42 is not applied to the endoscope 100 as a load. Accordingly, the endoscope 100 can be easily gripped.

(A-3) The pulsating flow generation unit 30 including the liquid chamber 42 does not need to move when the endoscopic device 10 is used, or does not need to be inserted into the tubular portion 110. Accordingly, size reduction is not required.

(B) The length L1 of the connection channel 53 is longer than the length L2 from the connection channel insertion port 130 to the distal end portion 120. Accordingly, an operator can grip the connection channel 53 in a state where the connection channel 53 is inserted into the tubular portion 110 and the ejecting tube 55 is disposed in the vicinity of the distal end portion 120.

(C) The connection channel 53 is designed so that the cross-sectional area of the channel is less likely to be changed even if the internal pressure is changed. Accordingly, the pulsating flow generated in the pulsating flow generation unit 30 is propagated to the ejecting tube 55 without being greatly attenuated. As a result, excision capability is further improved.

(D) The liquid supply channel 52 does not require design for restraining the pulsating flow from being attenuated. Accordingly, a material having the low Young's modulus may be used. The internal radius rᵢₙ may be increased. The external radius rₒᵤₜ may be decreased. If the Young's modulus is allowed to become lower, an inexpensive material such as polypropylene can be used as in the present embodiment. If the internal radius rᵢₙ increases, the channel resistance can be reduced. If the external radius rₒᵤₜ decreases, the wall thickness becomes thinner. Accordingly, weight reduction and cost reduction are realized, and flexibility is improved. Therefore, satisfactory usability is achieved.

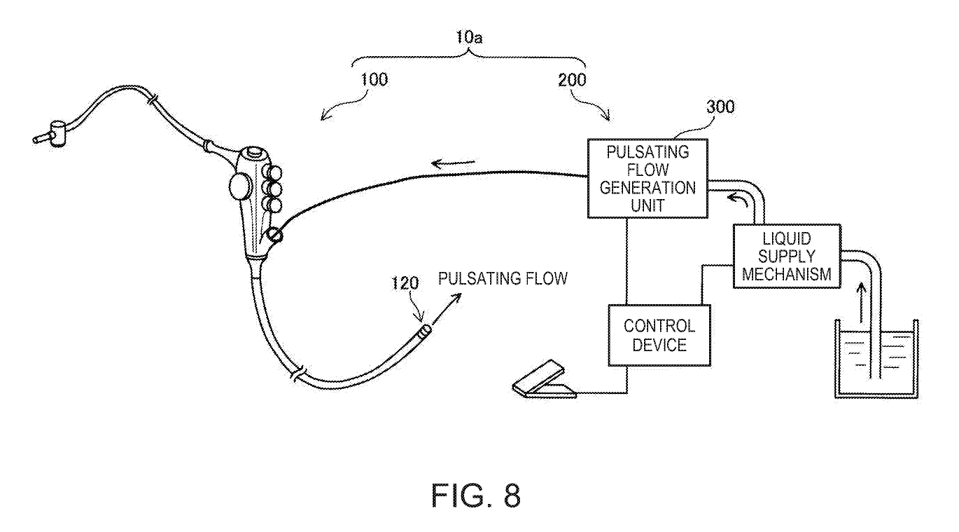

Embodiment 2 will be described. FIG. 8 is a configuration diagram of an endoscopic device 10a. The endoscopic device 10a is configured so that a liquid ejecting device 20a is combined with the endoscope 100. The endoscope 100 is the same as that described in Embodiment 1. The endoscopic device 10a has the same function as that of the endoscopic device 10 in Embodiment 1.

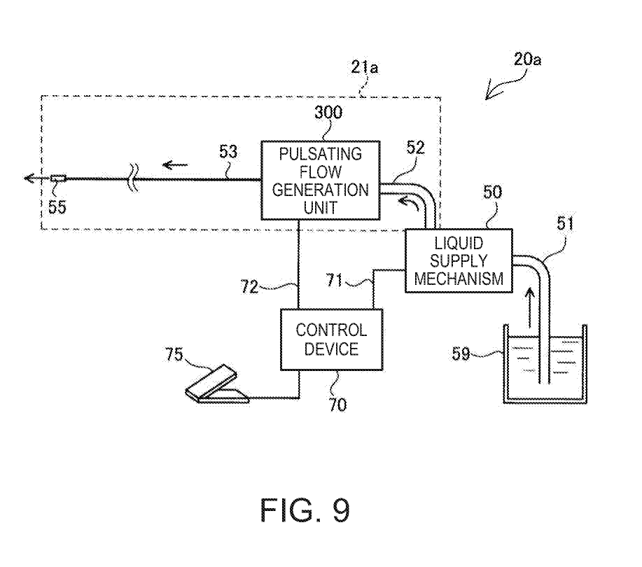

FIG. 9 illustrates the liquid ejecting device 20a. The liquid ejecting device 20a includes a liquid ejecting unit 21a instead of the liquid ejecting unit 21. The liquid ejecting unit 21a includes a pulsating flow generation unit 300 instead of the pulsating flow generation unit 30. Unless otherwise particularly described, other configuration elements are the same as those according to Embodiment 1.

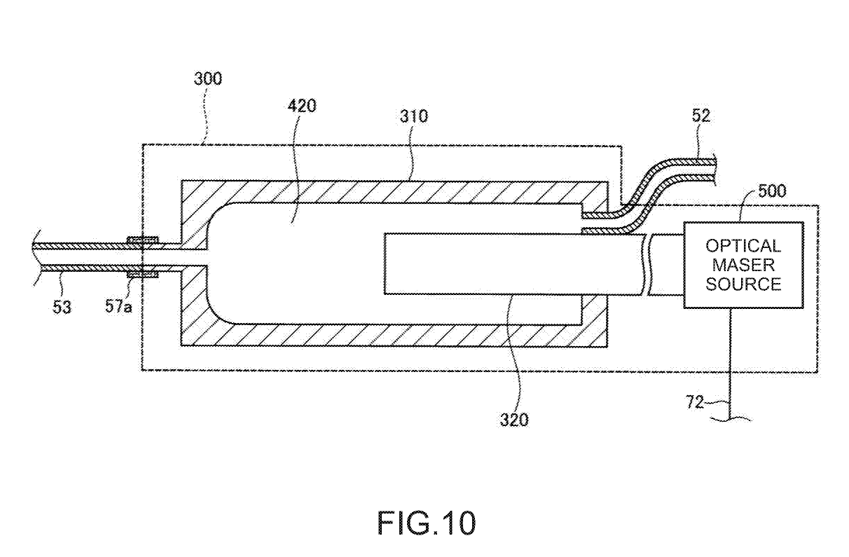

FIG. 10 is a sectional view of a liquid supply channel 52, a connection channel 53, and the pulsating flow generation unit 300. The pulsating flow generation unit 300 includes a pipe 310, an optical fiber 320, and an optical maser source 500. A liquid chamber 420 is formed inside the pipe 310 made of stainless steel. The liquid chamber 420 is connected to the liquid supply channel 52 and the connection channel 53.

The optical fiber 320 connects the inside of the liquid chamber 420 and the optical maser source 500 to each other. If a drive voltage is applied from the control device 70 via the signal line 72, the optical maser source 500 outputs an optical maser. The wavelength of the optical maser according to the present embodiment is 2.1 μm. The output optical maser is guided into the liquid chamber 420 by the optical fiber 320.

If the optical maser is discharged into the liquid chamber 420, the liquid in the vicinity of the distal end of the optical fiber 320 is vaporized after absorbing energy of the optical maser. In the present embodiment, the optical maser is intermittently output, and thus, the liquid is also intermittently vaporized. In this manner, air bubbles are intermittently generated in the vicinity of the distal end of the optical fiber 320. The intermittently generated air bubbles change the pressure in the liquid chamber 420. The pressure change generates the pulsating flow in the connection channel 53. Similarly to the liquid ejecting device 20 according to Embodiment 1, the pulsating flow can excise a lesion area. As described above, the optical maser source 500 functions as a pulsating flow generation unit and an air bubble generation portion. In addition, the liquid chamber 420 may be present as a place for generating the pulsating flow, or may be a portion of the liquid supply channel 52 or the connection channel 53. The shape or material is not limited thereto.

According to Embodiment 2, in a state where the liquid ejecting device 20a is combined with the endoscope 100, the pulsating flow generation unit 300 including the liquid chamber 420 is disposed outside the endoscope 100. Accordingly, it is also possible to obtain the advantageous effects described as (A) in Embodiment 1.

According to Embodiment 2, the liquid supply channel 52 and the connection channel 53 are disposed similarly to Embodiment 1. Accordingly, it is also possible to obtain the advantageous effects described as (B), (C), and (D) in Embodiment 1.

Without being limited to the embodiments, application examples, or modification examples described herein, the invention can be realized by adopting various configurations within the scope not departing from the gist of the invention. For example, in order to partially or entirely solve the above-described problems or in order to partially or entirely achieve the above-described advantageous effects, technical features in the embodiments, application examples, or modification examples corresponding to technical features in each aspect described in the Summary can be appropriately replaced or combined with each other. As long as it is not described herein that the technical features are essential, the technical features can be appropriately deleted. For example, the following configurations can be adopted as an example.

A configuration may be adopted so as to be capable of selecting any one between a mode for ejecting a large flow amount of liquid within a short time and a mode for ejecting a small flow amount of liquid for a long time. In a case where this configuration is used as a medical device, an operator may select the mode in accordance with a medical case.

A material of the ejecting tube, the connection channel, and the liquid supply channel may be appropriately changed. For example, the material maybe selected from metal or resins.

The endoscope may not be the above-described flexible type, and may be a rigid type.

The liquid to be ejected may be pure water or a drug solution.

Heating means used for the air bubble generation portion may not be the optical maser, and may be a resistor heater, a ceramic heater, or a microwave heater.

In the connection channel and the liquid supply channel, at least any one may be different among the Young's modulus, the outer diameter, the wall thickness, and the Poisson's ratio, and all of them may be the same as each other.

The pulsating flow generation unit including the liquid chamber may be disposed by being hung down in the vicinity of the endoscope, or may be hung down from the endoscope. Disposing in this way includes disposing the pulsating flow generation unit outside the endoscope in the present application. According to this configuration, the connection channel can be designed to be shorter. Therefore, it is possible to further restrain the pulsating flow from being attenuated.

Similarly to Embodiment 1, the liquid ejecting unit according to Embodiment 2 may be entirely replaced for every surgery, or may be partially replaced. For example, the optical maser source and the optical fiber may not be replaced. That is, when the pipe, the connection channel, and the liquid supply channel are replaced, the optical fiber may be attached to a new pipe from a used pipe. In this case, an expensive optical maser source can be much less frequently replaced.

The liquid ejecting unit may not be used in order to treat human diseases.

For example, the liquid ejecting unit may be used in order to treat animals' diseases other than the human diseases, or may be used in excising human body tissues for research or education.

Alternatively, the liquid ejecting unit may be used for a cleaning device which removes contamination by using an ejected liquid, or maybe used for a drawing device which draws a line by using the ejected liquid.

### REFERENCE SIGNS LIST

10 endoscopic device

10a endoscopic device

20 liquid ejecting device

20a liquid ejecting device

21 liquid ejecting unit

21a liquid ejecting unit

30 pulsating flow generation unit

31 pulsating flow generation unit

32 diaphragm

33 piezoelectric element

34 first case

36 second case

38 third case

39 reinforcement member

42 liquid chamber

45 outlet channel

50 liquid supply mechanism

51 channel

52 liquid supply channel

53 connection channel

55 ejecting tube

56 opening portion

57a connection ring

57b connection ring

59 liquid container

70 control device

71 signal line

72 signal line

75 foot switch

100 endoscope

110 tubular portion

113 flexible portion

115 bend portion

120 distal end portion

121 light

122 objective lens

123 air and water supply port

124 air and water intake port

125 opening portion

130 connection channel insertion port

140 grip portion

150 operation unit

160 connection portion

300 pulsating flow generation unit

310 pipe

320 optical fiber

420 liquid chamber

500 optical maser source

1. A liquid ejecting unit which is used in combination with an endoscope including an elongated tubular portion for being inserted into a body and a grip portion connected to the tubular portion, comprising:
a liquid chamber that is disposed outside the endoscope in a case where the liquid ejecting unit is combined with the endoscope;
a pulsating flow generation unit that generates a pulsating flow in the liquid chamber;
an opening portion for ejecting a liquid; and
a connection channel that is flexible, and that connects the liquid chamber and the opening portion to each other.

2. A liquid ejecting unit which is used in combination with an endoscope including an elongated tubular portion for being inserted into a body and a grip portion connected to the tubular portion, comprising:
a pulsating flow generation unit that generates a pulsating flow in a liquid chamber;
an opening portion for ejecting a liquid; and
a connection channel that is flexible, and that connects the liquid chamber and the opening portion to each other,
wherein a length of the connection channel is longer than a length of the tubular portion.

3. A liquid ejecting unit comprising:
a pulsating flow generation unit that generates a pulsating flow in a liquid chamber;
an opening portion for ejecting a liquid;
a connection channel that is flexible, and that connects the liquid chamber and the opening portion to each other; and
a liquid supply channel that functions as a channel for supplying a liquid to the liquid chamber, and that communicates with the liquid chamber,
wherein at least one of a Young's modulus, an outer diameter, a wall thickness, and a Poisson's ratio is different between the connection channel and the liquid supply channel.

4. The liquid ejecting unit according to claim 3,
wherein an increment of a cross-sectional area of a channel due to predetermined internal pressure loaded to the connection channel is smaller than an increment of a cross-sectional area of a channel due to the predetermined internal pressure loaded to the liquid supply channel.

5. The liquid ejecting unit according to claim 1,
wherein a volume of the liquid chamber can be changed, and
wherein the pulsating flow generation unit includes a volume change portion which can change the volume of the liquid chamber.

6. The liquid ejecting unit according to claim 1,
wherein the pulsating flow generation unit includes an air bubble generation portion for generating air bubbles inside the liquid chamber.

7.-9. (canceled)
